(19) United States
(12) Patent Application Publication
(10) Pub. No.: US 2022/0230743 A1
(43) Pub. Date: Jul. 21, 2022

(54) MEDICAL DEVICE MANAGEMENT USING RISK CONTROL MEASURES

(71) Applicant: Medtronic, Inc., Minneapolis, MN (US)

(72) Inventors: Todd D. Zenisek, Georgetown, TX (US); Touby A. Drew, Golden Valley, MN (US); Brian Andrew Smith, Apple Valley, MN (US); Juan G. Hincapie, Maple Grove, MN (US); Leonid M. Litvak, Los Angeles, CA (US); Zane K. Thimmesch-Gill, Minneapolis, MN (US)

(21) Appl. No.: 17/546,926

(22) Filed: Dec. 9, 2021

Related U.S. Application Data
(60) Provisional application No. 63/140,115, filed on Jan. 21, 2021.
(60) Provisional application No. 63/212,997, filed on Jun. 21, 2021.

(51) Int. Cl. G16H 40/67; H04W 4/20; G06F 21/62; G06N 20/00

(52) U.S. Cl. CPC G16H 40/67; G06N 20/00; G06F 21/6245; H04W 4/20

(57) ABSTRACT

A device comprising memory and processing circuitry coupled to the memory. The processing circuitry is configured to receive patient information for a medical device associated with the patient and select a communication mode for the patient based on the patient information. The processing circuitry is further configured to cause an output of the indication of the patient information using the selected communication mode.

This application claims priority to U.S. Provisional Patent Application No. 63/140,115, filed Jan. 21, 2021 and U.S. Provisional Patent Application No. 63/212,997, filed Jun. 21, 2021, each of which is incorporated herein by reference.

### TECHNICAL FIELD

This disclosure generally relates to medical devices.

### BACKGROUND

Medical devices (e.g., an implantable medical device or an external medical device) may include electrical stimulation devices, drug pumps, insulin pumps, or cardiac stimulation devices. Electrical stimulation devices, for example, neurostimulators or neurostimulation devices, may be external to or implanted within a patient, and configured to deliver electrical stimulation therapy to various tissue sites to treat a variety of symptoms or conditions such as chronic pain, tremor, Parkinson's disease, epilepsy, or other neurological disorders, urinary or fecal incontinence, sexual dysfunction, obesity, or gastroparesis. An electrical stimulation device may deliver electrical stimulation therapy via electrodes, e.g., carried by one or more leads, positioned proximate to target locations associated with the brain, the spinal cord, pelvic nerves, tibial nerves, peripheral nerves, the gastrointestinal tract, or elsewhere within a patient. Stimulation proximate the spinal cord, proximate the sacral nerve, within the brain, and proximate peripheral nerves is often referred to as spinal cord stimulation (SCS), sacral neuromodulation (SNM), deep brain stimulation (DBS), and peripheral nerve stimulation (PNS), respectively.

### SUMMARY

In general, this disclosure describes techniques for communication of patient information for a patient receiving treatment by a clinician. Medical devices managed by a clinician may generate patient information to help the clinician review an operating status of the medical devices. The medical devices may be implantable and/or wearable and may be configured to provide one or more of deep brain stimulation (DB S), spinal cord stimulation (SCS), sacral neuromodulation (SNM), and peripheral nerve stimulation (PNS), targeted drug delivery (TDD), or another therapy. For example, patient information for an implantable fluid delivery device may indicate one or more of a current reservoir status, a projected refill status, a device replacement status, a patient adjustment, a patient activity, a patient input, a sensed signal, or a device operational status. A clinician device may display a complete listing (e.g., a spreadsheet or “snapshot”) of patient information for all of the clinician's patients. The clinician may determine an efficacy of treatment and/or improve a therapy for the clinician's patients using the complete listing of all of the patient information.

The techniques of this disclosure may include a device (e.g., an external programmer and/or a cloud) configured to select a communication mode based on patient information. For example, one or more processors arranged in an external clinician programmer device and/or a cloud (e.g., using a web interface of the cloud) may be configured to select the communication mode to include one or more of a browser presentation, an e-mail, a text message, a telephone call, a virtual visit, or an in-person visit. Approval of therapy changes for some patients may be voluntary or based on website, email or text communication with a caregiver, whereas other patients may require live voice or video communication. In this way, a system may help to provide risk control measures that simplify or make changes to a therapy easier for low risk changes using simple and/or non-intrusive communication modes (e.g., an e-mail, a text message, an in-app notification, etc.) and that may alert or prioritize changes to a therapy that may be relatively risky and would most likely benefit from a clinician review using interactive communication modes (e.g., a telephone call, a virtual visit, an in-person visit, etc.), which may improve a therapy provided to the patient. Moreover, providing risk control measures using a communication mode may allow a clinician to more quickly review therapy changes, which may help to reduce an amount of time a clinician spends reviewing patient information.

In one example, a method for managing communications with a patient includes receiving, by one or more processors, patient information for a medical device associated with patient and selecting, by the one or more processors, a communication mode for the patient based on the patient information. The method further includes causing, by the one or more processors, an output of the indication of the patient information using the selected communication mode.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) in the form of a neurostimulation device configured to deliver spinal cord stimulation (SCS) and an external programmer, in accordance with one or more techniques of this disclosure.

FIG. 2 is a block diagram illustrating an example of an IMD in the form of a stimulation device, in accordance with one or more techniques of this disclosure.

FIG. 3 is a block diagram illustrating an example of an external programmer suitable for use with the IMD of FIG. 2, in accordance with one or more techniques of this disclosure.

FIG. 4 is a block diagram illustrating an example of one or more remote servers and one or more remote clients suitable for use with the IMD of FIG. 1, in accordance with one or more techniques of this disclosure.

FIG. 5 is a flow diagram illustrating a process for selecting a communication mode to output patient information, in accordance with one or more techniques of this disclosure.

### DETAILED DESCRIPTION

Efficacy of treatment (e.g., stimulation, drug delivery, etc.) in eliminating or alleviating symptoms, preventing or delaying onset or progression of aspects of, or restoring functions impaired or diminished by a disease, disorder, syndrome or injury, may vary according to the parameters used to deliver the treatment to a patient. Selection of electrode positions relative to a neural target, as one example, can elicit a desired response to the stimulation. Delivering stimulation with different stimulation parameters, such as different electrodes, electrode combinations and/or polarities, or different stimulation amplitudes, pulse widths, pulse rates, or cycling can result in differences in efficacy for a variety of therapies such as, for example, spinal cord stimulation (SCS) to relieve pain or restore physical function or control in the case of spinal cord injury or degeneration.

The techniques of this disclosure may include a device (e.g., an external programmer and/or a remote client) configured to select a communication mode based on patient information. For example, one or more processors may be configured to select the communication mode to include one or more of a browser presentation, an e-mail, a text message, a telephone call, a virtual visit, or an in-person visit. Approval of therapy changes for some patients may be voluntary or based on website, email or text communication with a caregiver, whereas other patients may require live voice or video communication. Live communication may be more important for targeted drug delivery (TDD) therapies. Live voice or video communication may comprise a time-stamped audio or video snippets. In this way, the one or more processors may initiate a communication with the patient using a communication mode that may help to reduce an amount of time for the clinician review a treatment of patients.

For example, the one or more processors may determine a severity level for patient information and select the communication mode based on the severity level, e.g., according to an escalation protocol that specifies a given communication mode for a corresponding severity level. For instance, the one or more processors may select an in-person visit for making adjustments for a TDD therapy and may select a text message for making adjustments for a SCS therapy. As another example, the one or more processors may require an in-person visit or video conference to approve an increase in drug dosage, and may select a text chat for evaluating a patient to reduce a drug dosage.

Techniques described herein may be directed to implantable medical devices and external medical devices. Examples described herein may describe techniques with reference to medical devices, however, aspects of such techniques may apply to any medical device. Again, examples of medical devices, which may be external or implantable), may include drug pumps, insulin pumps, cardiac stimulation devices, and neurostimulation devices, as well as programmers or controllers that communicate with drug pumps, insulin pumps, cardiac stimulation device, and neurostimulation devices and cloud-based server devices that communicate with programmers or controllers or with drug pumps, insulin pumps, cardiac stimulation devices, and neurostimulation devices.

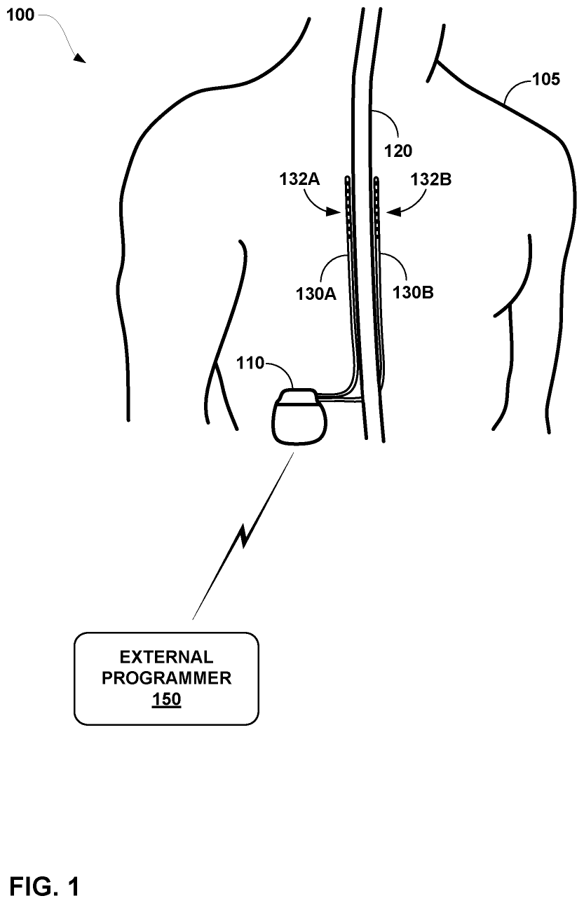

FIG. 1 is a conceptual diagram illustrating an example system 100 that includes an implantable medical device (IMD) 110 configured to deliver SCS therapy, processing circuitry 140, and an external programmer 150, in accordance with one or more examples of this disclosure. External programmer 150 may use a communication mode that is selected based on patient information, which may help to prioritize a delivery of data between patient 105 and a clinician. Although the examples described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of stimulation.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105, e.g., for relief of chronic pain or other symptoms, or restoration or support of physical function or control in the case of spinal cord injury or degeneration, via one or more electrodes 132A, 132B of leads 130A and/or 130B, respectively. In the example of FIG. 1, each lead 130A, 130B includes eight electrodes 132A, 132B respectively, although the leads may each have a different number of electrodes. Leads 130A, 130B may be referred to collectively as “leads 130” and electrodes 132A, 132B may be referred to collectively as electrodes 132. In other examples, 1 MB 110 may be coupled to a single lead carrying multiple electrodes or more than two leads each carrying multiple electrodes.

IMD 110 may be a chronic electrical stimulator that remains implanted within patient 105 for weeks, months, or years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to one or more leads percutaneously implanted within the patient. In some examples, 1 MB 110 uses electrodes on one or more leads, while in other examples, 1 MB 110 use one or more electrodes on a lead or leads and one of more electrodes on a housing of the IMD. In further examples, IMD 110 may be leadless and instead use only electrodes carried on a housing of IMD.

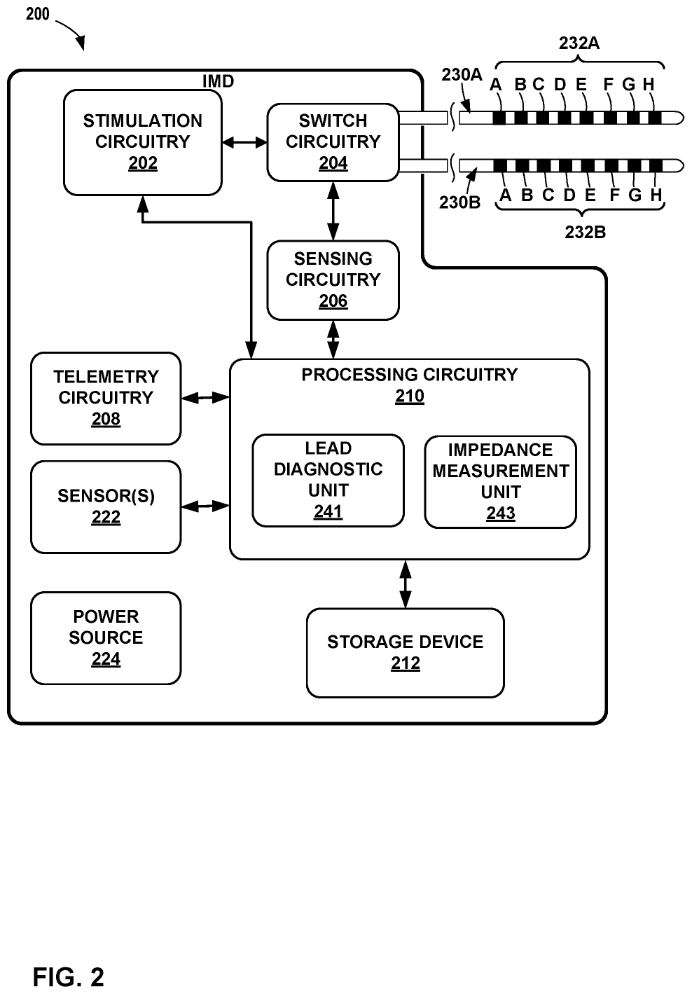

IMD 110 may be constructed of any polymer, metal, or composite material sufficient to house the components of IMD 110 (e.g., components illustrated in FIG. 2) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted at other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

In the example of FIG. 1, electrical stimulation energy, which may be delivered as regulated current or regulated voltage-based pulses, is delivered from IMD 110 to one or more target tissue sites of patient 105 via leads 130 and electrodes 132. Leads 130 position electrodes 132 adjacent to target tissue of spinal cord 120. One or more of the electrodes 132 may be disposed at a distal tip of a lead 130 and/or at other positions at intermediate points along the lead. Leads 130 may be implanted and coupled to IMD 110. The electrodes 132 may transfer electrical stimulation generated by an electrical stimulation generator in IMD 110 to tissue of patient 105. Although leads 130 may each be a single lead, a lead 130 may include a lead extension or other segments that may aid in implantation or positioning of lead 130.

The electrodes 132 of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration. Deployment of electrodes via leads 130 is described for purposes of illustration, but electrodes may be arranged on a housing of IMD 110, e.g., in rows and/or columns (or other arrays or patterns), as surface electrodes, ring electrodes, or protrusions.

Stimulation parameters defining the electrical stimulation pulses delivered by IMD 110 through electrodes 132 of leads 130 may include information identifying which electrodes have been selected for delivery of the stimulation pulses according to a stimulation program and the polarities of the selected electrodes (the electrode combination), and voltage or current amplitude, pulse rate (e.g., frequency), and pulse width of the stimulation pulses. The stimulation parameters may further include a cycle parameter that specifies when, or how long, stimulation is turned on and off. Stimulation parameters may be programmed prior to delivery of the stimulation pulses, manually adjusted based on user input, or automatically controlled during delivery of the stimulation pulses, e.g., based on sensed conditions.

Although the example of FIG. 1 is directed to SCS therapy, e.g., to treat pain or restore or support physical function or control in the case of spinal cord injury or degeneration, in other examples, system 100 may be configured to treat other conditions that may benefit from stimulation therapy. For example, system 100 may be used to treat tremor, Parkinson's disease, epilepsy, or other neurological disorders, urinary or fecal incontinence, sexual dysfunction, obesity, or gastroparesis, or psychiatric disorders such as depression, mania, obsessive compulsive disorder, or anxiety disorders. Hence, in some examples, system 100 may be configured to deliver SNM, DBS, PNS, or other stimulation, such as peripheral nerve field stimulation (PNFS), cortical stimulation (CS), gastrointestinal stimulation, or any other stimulation therapy capable of treating a condition of patient 105.

Leads 130 may include, in some examples, one or more sensors configured to sense one or more physiological parameters of patient 105, such as patient activity, pressure, temperature, or other characteristics. At least some of electrodes 132 may be used to sense electrical signals within patient 105, additionally or alternatively to delivering stimulation. IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation. In some examples, the target tissue includes nerves, smooth muscle or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical or lumbar regions.

Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may reduce the perception of pain by patient 105, and thus, provide efficacious therapy results. In some examples, some electrical stimulation pulses may be directed to glial cells while other electrical stimulation (e.g., delivered by a different electrode combination) is directed to neurons. In other examples, electrical stimulation pulses may be directed to restore a function lost due to a spinal cord injury.

IMD 110 may generate and may deliver electrical stimulation therapy to a target stimulation site within patient 105 via the electrodes of leads 130 to patient 105 according to one or more therapy stimulation programs. A therapy stimulation program specifies values for one or more parameters that define an aspect of the therapy delivered by IMD 110 according to that program. For example, a therapy stimulation program that controls delivery of stimulation by IMD 110 in the form of stimulation pulses may define values for voltage or current pulse amplitude, pulse width, and pulse rate (e.g., pulse frequency) for stimulation pulses delivered by IMD 110 according to that program, as well as the particular electrodes and polarities forming an electrode combination used to deliver the stimulation pulses.

A user, such as a clinician, caretaker, or patient 105, may interact with a user interface of an external programmer 150 to program IMD 110. External programmer 150 may represent a physician programmer or patient programmer. Programming of IMD 110 may refer generally to the generation and transfer of commands, programs, or other information to control the operation of IMD 110. In this manner, IMD 110 may receive the transferred commands and programs from external programmer 150 to control electrical stimulation therapy. External programmer 150 may transmit therapy stimulation programs, program groups, stimulation parameter adjustments, therapy stimulation program selections, user input, or other information to control the operation of IMD 110, e.g., by wireless telemetry or wired connection.

External programmer 150 may perform a stimulation parameter adjustment that changes a set of stimulation parameters of an existing program. For example, external programmer 150 may automatically, semi-automatically, or based on a user selection, may determine or more stimulation parameter adjustments for an existing program. In this example, external programmer 150 may pass through the one or more parameter adjustments for the existing program. For instance, external programmer 150 may determine a parameter adjustment (e.g., receive the adjustment from a user input from a health professional) that sets an intensity value of a particular stimulation parameter of a program and may relay the parameter adjustment to IMD 110.

External programmer 150 may be characterized as a physician or clinician programmer if external programmer 150 is primarily intended for use by a physician or clinician. In other cases, external programmer 150 may be characterized as a patient programmer if external programmer 150 is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change stimulation therapy. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

IMD 110 and external programmer 150 may exchange information and may communicate via wireless communication. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

IMD 110, in response to commands from external programmer 150, may deliver electrical stimulation therapy according to one or more therapy stimulation programs, or a group of programs to a target tissue site of the spinal cord 120 of patient 105 via electrodes 132 on leads 130. In some examples, IMD 110 automatically modifies therapy stimulation programs as therapy needs of patient 105 evolve over time. For example, the modification of the therapy stimulation groups or programs may cause the adjustment of at least one parameter of the plurality of stimulation pulses.

In accordance with the techniques of the disclosure, external programmer 150 may be configured to determine patient information for a clinician to review. While FIG. 1 shows one programmer (e.g., external programmer 150), some examples may include additional and/or alternative programmers. For example, a system may include a patient programmer and a clinician programmer. The patient may interact with the patient programmer to select pain level, a patient activity level, a side effect rating, and/or initiate a patient adjustment. The clinician programmer may receive the information from the patient programmer (e.g., directly or through a networked communication link). External programmer 150 may generate the patient information based on input by patient 105. For instance, patient 105 may interact with a user interface of external programmer 150 to select a pain level, a patient activity level, a side effect rating, and/or initiate a patient adjustment. In some examples, external programmer 150 may automatically or semi-automatically generate the patient information. For example, external programmer 150 may generate the patient information using an output from one or more sensors (e.g., one or more accelerometers and/or one or more gyroscopes) arranged on external programmer 150.

In some examples, external programmer 150 may generate the patient information based on information from IMD 110. For example, external programmer 150 may receive an output from IMD 110 indicating a device operational status. The device operation status may indicate, for example, a battery level of IMD 110. Examples of device operation status may include, for example, a time recharging, a time utilized by group of programs or a program, events that may have occurred during a given time, or a battery status.

The neurological signals sensed within a brain of patient 105 may reflect changes in electrical current produced by the sum of electrical potential differences across brain tissue. Examples of neurological brain signals include, but are not limited to, bioelectric signals generated from local field potentials (LFP) sensed within one or more regions of brain 120. Electroencephalogram (EEG) signal or an electrocorticogram (ECoG) signal are also examples of bioelectric signals. For example, neurons generate the bioelectric signals, and if measured at depth, it is LFP, if measured on the coretex, it is ECoG, and if on scalp, it is EEG. In this disclosure, the term “oscillatory signal source” is used to describe a signal source that generates bioelectric signals.

One example of the feature of interest (e.g., biomarker) within the LFPs is synchronized beta frequency band (13-33 Hz) LFP activity recorded within the sensorimotor region of the subthalamic nucleus (STN) in Parkinson's disease patients. The source of the LFP activity can be considered as an oscillatory signal source, within the brain of the patient, that outputs an oscillatory electrical voltage signal that is sensed by one or more of electrodes 116 and/or 118. The suppression of pathological beta activity (e.g., suppression or squelching of the signal component of the bioelectric signals generated from the oscillatory LFP signal source that is within the beta frequency band) by both medication and DBS may correlate with improvements in the motor symptoms of patients who have Parkinson's disease.

ECAPs are a measure of neural recruitment because each ECAP signal represents the superposition of electrical potentials generated from a population of axons firing in response to an electrical stimulus (e.g., a stimulation pulse). Changes in a characteristic (e.g., an amplitude of a portion of the signal or area under the curve of the signal) of an ECAP signals occur as a function of how many axons have been activated by the delivered stimulation pulse. For a given set of parameter values that define the stimulation pulse and a given distance between the electrodes and target nerve, the detected ECAP signal may have a certain characteristic value (e.g., amplitude). Therefore, a system can determine that the distance between electrodes and nerves has increased or decreased in response to determining that the measured ECAP characteristic value has increased or decreased. For example, if the set of parameter values stays the same and the ECAP characteristic value of amplitude increases, the system can determine that the distance between electrodes and the nerve has decreased.

External programmer 150 may receive an output from IMD 110 indicating one or more sensed signals. Sensed signals from IMD 110 may include, for example, one or more of an electrocardiogram, a breathing rate, evoked potential (e.g., an ECAP), Electromyography (EMG), or local field potential (LFP).

In accordance with the techniques of the disclosure, external programmer 150 may cause IMD 110 to initiate a diagnostic test. Examples of a diagnostic test may include, for example, a lead location diagnostic, an impedance measurement, and/or sensing an ECAP signal. For example, a remote device (e.g., a remote server or remote client) may cause external programmer 150 to initiate the diagnostic test. In some examples, however, external programmer 150 may initiate the diagnostic test without the remote device.

External programmer 150 may request a patient input. For example, in response to an instruction from a remote device (e.g., a remote server or remote client), external programmer 150 may output a request for patient 105 to input one or more of a pain rating, a side effect rating, or a confirmation of a patient activity level. The patient activity level may include, for example, standing, walking, laying down, or voiding. External programmer 150 may output patient information automatically, semi-automatically, or manually generated using input by patient 105 to a remote device (e.g., a remote server or remote client).

In accordance with the techniques of the disclosure, external programmer 150 or another device (e.g., a remote device) may determine a communication mode. Examples of communication modes may include, for example, one or more of a browser presentation, an e-mail, a text message, a call, a virtual visit, a video message, a voice message, or an in-person visit. External programmer 150 may determine the communication mode based on patient information.

External programmer 150 may determine a severity level for patient information. For example, each communication mode of a plurality of communication modes may be assigned a respective range of severity levels. In this example, external programmer 150 may select the communication mode that comprises a range of severity level that includes the severity level for the patient information. For instance, patient information may be associated with a severity level of 3, which may be assigned to a text message. In this instance, external programmer 150 may select a text message as a communication mode for the patient information. External programmer 150 may output patient information using a communication mode. In this way, external programmer 150 may help to provide risk control measures that simplify or make changes to a therapy easier for low risk changes using simple and/or non-intrusive communication modes (e.g., an e-mail, a text message, an in-app notification, etc.) and that may alert or prioritize changes to a therapy that may be relatively risky and would most likely benefit from a clinician review using relatively complex communication modes (e.g., a telephone call, a virtual visit, an in-person visit, etc.), which may improve a therapy provided to the patient. Moreover, providing risk control measures using a communication mode may allow a clinician to more quickly review therapy changes, which may help to reduce an amount of time a clinician spends reviewing patient information.

Patient information may be associated (e.g., assigned) one or more tags that identify a severity level for the respective patient information. The tags may be pre-configured, configured by a clinician, and/or determined by one or more of external programmer 150 or a remote server. For example, a clinician may select a severity level for types of patient information (e.g., all caution). In some examples, a clinician may silence, decrease, or increase a severity determined by implant (e.g., recommendation of severity). One or more of external programmer 150 or a remote server may determine the severity level for patient information based on feedback from a user (e.g., patient 105 or caretaker of patient 105). For instance, one or more of external programmer 150 or a remote server may apply a learning algorithm (e.g., a machine learning algorithm) to determine the severity level for patient information that is trained using feedback from the user. The feedback may comprise a patient reported outcome (PRO). For instance, external programmer 150 may generate the feedback using an input responsive to a prompting of patient 150 or a caretaker of patient 150. In some examples, external programmer 150 may determine the feedback based on one or more user interactions with external programmer 150. For instance, external programmer 150 may determine that patient 150 is not satisfied with a therapy provided by IMD 110 in response to receiving an indication to increase a stimulation value for IMD 110.

Tags may indicate a subset of a plurality of severity levels, such as, for example, an information level, a caution level, and a high severity level. While information, caution, and high severity levels are used in the following examples, other severity levels may be used and/or some severity levels may be omitted. A caution severity may indicate that associated patient information may be dangerous.

IMD 110 may generate sensor information for an event. In this example, one or more of IMD 110 or external programmer 150 may determine that the sensor information exceeds a threshold. In this example, one or more of IMD 110 or external programmer 150 may determine the severity level based on a tag associated with the sensor information. For example, one or more of IMD 110 or external programmer 150 may select a communication mode for an information level an amount of time (e.g., a week) prior from when the IMD 110 is expected to be low of medication to allow time to order medication. In some examples, one or more of IMD 110 or external programmer 150 may select a communication mode for an information severity level when IMD 110 and/or external programmer 150 determines that more medication has been used by IMD 110 than estimated (e.g., a rate or usage of the medication is greater than a threshold).

One or more of IMD 110 or external programmer 150 may select a communication mode for a caution severity level when IMD 110 detects a low reservoir status (e.g., a low reservoir alarm is triggered). IMD 110 may detect a low reservoir status when an amount of medication is less than a low threshold. One or more of IMD 110 or external programmer 150 may select a communication mode for a high severity level when IMD 110 detects that a reservoir is empty. IMD 110 may detect an empty reservoir status when an amount of medication is less than an empty threshold.

One or more of IMD 110 or external programmer 150 may select a communication mode for a high severity level in response to a determination that one or more refills of a reservoir of IMD 110 have been missed. For example, external programmer 150 may determine that one or more refills of a reservoir of IMD 110 have been missed when IMD 110 does not indicate an increase in medication in a reservoir of IMD 110 in response to a notification to refill the reservoir. While the above examples refer to a reservoir for medication, some examples, may use other types of storage, for example, energy stored by an energy storage device (e.g., a capacitor, a flywheel, or a battery). For example, one or more of IMD 110 or external programmer 150 may select a communication mode based on a status of a battery of IMD 110.

FIG. 2 is a block diagram illustrating an example configuration of components of an IMD 200, in accordance with one or more techniques of this disclosure. IMD 200 may be an example of IMD 110 of FIG. 1. In the example shown in FIG. 2, IMD 200 includes stimulation generation circuitry 202, switch circuitry 204, sensing circuitry 206, telemetry circuitry 208, processing circuitry 210, storage device 212, sensor(s) 222, power source 224, lead 230A carrying electrodes 232A, which may correspond to lead 130B and electrodes 132B of FIG. 1, and lead 230B carrying electrodes 232B, which may correspond to lead 130B and electrodes 132B of FIG. 1.

Stimulation generation circuitry 202 may generate electrical stimulation pulses selected to alleviate symptoms or dysfunction of one or more diseases, disorders, injuries, or syndromes. Intensity may be a function of amplitude, pulse width, and/or frequency of the electrical stimulation pulses. While stimulation pulses are described, stimulation signals may take other forms, such as continuous-time signals (e.g., sine waves) or the like. Each of leads 230A, 230B may include any number of electrodes 232A, 232B. In the example of FIG. 2, each set of electrodes 232A, 232B includes eight electrodes A-H. In some examples, the electrodes are arranged in bipolar combinations. A bipolar electrode combination may use electrodes carried by the same lead 230A, 230B or different leads. For example, an electrode A of electrodes 232A may be a cathode and an electrode B of electrodes 232A may be an anode, forming a bipolar combination.

Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232A, 232B, or directed sensed signals from one or more of electrodes 232A, 232B to sensing circuitry 206. In some examples, each of the electrodes 232A, 232B may be associated with respective regulated current source and sink circuitry to selectively and independently configure the electrode to be a regulated cathode or anode, in which case switch circuitry 204 may not be necessary to direct stimulation signals to electrodes. Instead, current sourced or sunk by selected electrodes may be individually controlled. Stimulation generation circuitry 202 and/or sensing circuitry 206 also may include sensing circuitry to direct electrical signals sensed at one or more of electrodes 232A, 232B.

Sensing circuitry 206 may be configured to monitor signals from any combination of electrodes 232A, 232B. In some examples, sensing circuitry 206 includes one or more amplifiers, filters, and analog-to-digital converters. Sensing circuitry 206 may be used to sense electrophysiological signals. In some examples, sensing circuitry 206 detects electrophysiological signals from a particular combination of electrodes 232A, 232B. In some cases, the particular combination of electrodes for sensing electrophysiological signals includes different electrodes than a set of electrodes 232A, 232B used to deliver stimulation pulses. Alternatively, in other cases, the particular combination of electrodes used for electrophysiological sensing includes at least one of the same electrodes as a set of electrodes used to deliver stimulation pulses to patient 105. Sensing circuitry 206 may provide signals to an analog-to-digital converter, for conversion into a digital signal for processing, analysis, storage, or output by processing circuitry 210.

Telemetry circuitry 208 may support wireless communication between IMD 200 and an external programmer (not shown in FIG. 2) or another computing device under the control of processing circuitry 210. Processing circuitry 210 of IMD 200 may receive, as updates to programs, values for various stimulation parameters such as amplitude and electrode combination, from the external programmer via telemetry circuitry 208. Telemetry circuitry 208 in IMD 200, as well as telemetry circuits in other devices and systems described herein, such as the external programmer, may accomplish communication by radiofrequency (RF) communication techniques. In addition, telemetry circuitry 208 may communicate with an external medical device programmer (not shown in FIG. 2) via proximal inductive interaction of IMD 200 with the external programmer. The external programmer may be one example of external programmer 150 of FIG. 1. Accordingly, telemetry circuitry 208 may send information to the external programmer on a continuous basis, at periodic intervals, or upon request from IMD 110 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. As shown, processing circuitry 210 may comprise a lead diagnostic unit 241, and an impedance measurement unit 243 that may each comprise circuitry and/or software instructions. The software instructions associated with lead diagnostic unit 241, and impedance measurement unit 243 may be stored, for example, at storage device 212.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210, such as, for example, instructions associated with lead diagnostic unit 241, and impedance measurement unit 243.

Power source 224 may be configured to deliver operating power to the components of IMD 200. Power source 224 may include a battery and a power generation circuit to produce the operating power. In some examples, the battery is rechargeable to allow extended operation. In some examples, power source 224 may be configured to recharge a battery through proximal inductive interaction between an external charger and an inductive charging coil within IMD 200. Power source 224 may include any one or more of a plurality of different battery types, such as nickel cadmium batteries and lithium ion batteries.

Telemetry circuitry 208 may process a request from external programmer 150 to initiate a diagnostic test. For example, lead diagnostic unit 241 of processing circuitry 210 may perform a lead location diagnostic of a lead of leads 230 in response to the request to initiate the diagnostic test. For example, lead diagnostic unit 241 may track a lead tip movement in relation to stimulation changes (e.g., a lead or an impedance issue) or lead location check. Lead diagnostic unit 230 may monitor lead tip movement by, for example, monitoring impedance changes between electrodes on separate leads. In response to impedance changes above a threshold between relatively placed electrodes, lead diagnostic unit 230 may determine that the electrodes may have moved. Lead diagnostic unit 230 may measure impedance changes between any of several electrodes on one lead to any of several electrodes on the other lead. Lead diagnostic unit 230 may measure impedance changes between multiple combinations of electrodes between separate leads. Movement of a lead 230 may result in therapy being provided in a different part of patient 105, which may reduce an effectiveness of therapy provided by IMD 110 to patient 105. Testing for the movement of a lead of leads 230 may help to identify when a patient would benefit from a review by the clinician. For example, a clinician may set an importance attribute for a lead position difference to prioritize patient information indicating that at least one lead of leads 230 moves more than a threshold value.

In some examples, impedance measurement unit 243 of processing circuitry 210 may perform an impedance measurement of a lead of leads 230 in response to the request to initiate the diagnostic test. A change in impedance of a lead of leads 230 may indicate a reduction in an effectiveness of therapy provided by IMD 110 to patient 105. Testing for the impedance of leads 230 may help to identify when a patient would benefit from a review by the clinician. For example, a clinician may set an importance attribute for a lead impedance difference to prioritize patient information indicating that at least one lead of leads 230 changes in impedance more than a threshold value.

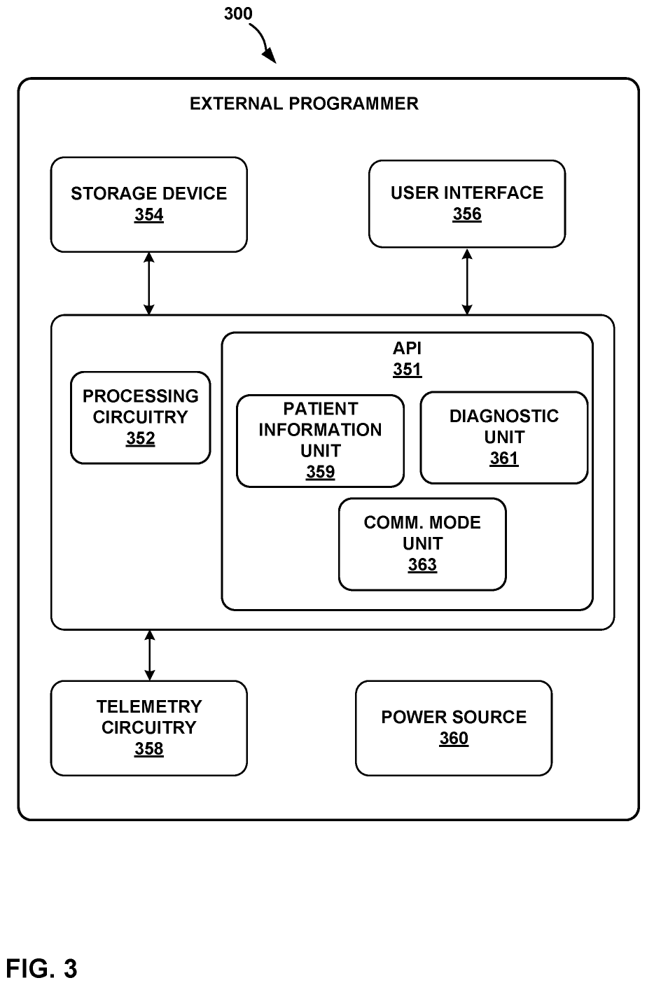

FIG. 3 is a block diagram illustrating an example configuration of components of an example external programmer 300. External programmer 300 may be an example of external programmer 150 (e.g., an external patient programmer or an external clinician programmer) of FIG. 1. Although external programmer 300 may generally be described as a hand-held device, external programmer 300 may be a larger portable device or a more stationary device. In addition, in other examples, external programmer 300 may be included as part of an external charging device or include the functionality of an external charging device. As illustrated in FIG. 3, external programmer 300 may include processing circuitry 352, storage device 354, user interface 356, telemetry circuitry 358, and power source 360. Storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include one or more processors, such as, one or more microprocessors, DSPs, ASICs, FPGAs, or any other equivalent integrated or discrete logic circuitry, as well as any combinations of such components, configured to perform the processes discussed with respect to processing circuitry 352. External programmer 300 may represent a patient programmer, clinician programmer, or another device.

In general, external programmer 300 includes any suitable arrangement of hardware, alone or in combination with software and/or firmware, to perform the techniques attributed to external programmer 300, and processing circuitry 352, user interface 356, and telemetry circuitry 358 of external programmer 300. In various examples, external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and telemetry circuitry 358 are described as separate modules, in some examples, processing circuitry 352 and telemetry circuitry 358 are functionally integrated. In some examples, processing circuitry 352 and telemetry circuitry 358 correspond to individual hardware units, such as ASICs, DSPs, FPGAs, or other hardware units.

Storage device 354 (e.g., a storage device) may store instructions that, when executed by processing circuitry 352, cause processing circuitry 352 and external programmer 300 to provide the functionality ascribed to external programmer 300 throughout this disclosure. For example, storage device 354 may include instructions that cause processing circuitry 352 to obtain a parameter set from memory or receive user input and send a corresponding command to IMD 110, or instructions for any other functionality. In addition, storage device 354 may include a plurality of programs, where each program includes a parameter set that defines therapy stimulation or control stimulation. Storage device 354 may also store data received from a medical device (e.g., IMD 110). For example, storage device 354 may store data recorded at a sensing module of the medical device, and storage device 354 may also store data from one or more sensors of the medical device.

Processing circuitry 352 may be configured to control IMD 110 with a program to provide stimulation. For example, processing circuitry 352 may automatically or semi-automatically set or adjust programs at IMD 110 by transmitting, with telemetry circuitry 358, instructions to IMD 110. For instance, in response to a change (e.g., a change indicated by user input, a change sensed by IMD 110, etc.) in activity of a patient (e.g., standing, walking, voiding, etc.), processing circuitry 352 may automatically or semi-automatically set or adjust programs at IMD 110. For instance, processing circuitry 352 may, in response to determining that the patient would not like to void, output instructions to IMD 110 to use a first group stored at IMD 110 for controlled voiding. In this instance, processing circuitry 352 may, in response to determining that the patient would like to void, output instructions to IMD 110 to use a new group or program stored at IMD 110 for controlled voiding.

User interface 356 may include a button or keypad, lights, a speaker for voice commands, a display, such as a liquid crystal (LCD), light-emitting diode (LED), or organic light-emitting diode (OLED). In some examples the display includes a touch screen. User interface 356 may be configured to display any information related to the delivery of electrical stimulation. User interface 356 may also receive user input (e.g., indication of when the patient perceives a stimulation pulse) via user interface 356. The input may be, for example, in the form of pressing a button on a keypad or selecting an icon from a touch screen. The input may request starting or stopping electrical stimulation, the input may request a new spatial electrode pattern or a change to an existing spatial electrode pattern, of the input may request some other change to the delivery of electrical stimulation.

Telemetry circuitry 358 may support wireless communication between the medical device and external programmer 300 under the control of processing circuitry 352. Telemetry circuitry 358 may also be configured to communicate with another computing device via wireless communication techniques, or direct communication through a wired connection. In some examples, telemetry circuitry 358 provides wireless communication via an RF or proximal inductive medium. In some examples, telemetry circuitry 358 includes an antenna, which may take on a variety of forms, such as an internal or external antenna.

Examples of local wireless communication techniques that may be employed to facilitate communication between external programmer 300 and IMD 110 include RF communication according to the 802.11 or Bluetooth® specification sets or other standard or proprietary telemetry protocols. In this manner, other external devices may be capable of communicating with external programmer 300 without needing to establish a secure wireless connection. As described herein, telemetry circuitry 358 may be configured to transmit a spatial electrode movement pattern or other stimulation parameter values to IMD 110 for delivery of electrical stimulation therapy.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

Processing circuitry 352 may implement API 351 to facilitate the control of IMD 110. API 351 may include patient information unit 359, diagnostic unit 361, and communication mode unit 363. Patient information unit 359 may automatically, semi-automatically, or manually generate patient information using, for example, sensed data, patient input, or other information. For example, patient information unit 359 may output, with telemetry circuitry 358, patient information to a remote device and/or a remote client.

Diagnostic unit 361 may be configured to cause IMD 110 to perform one or more diagnostic tests. For example, diagnostic unit 361 may cause IMD 110 to perform one or more of a lead location diagnostic for a plurality of leads inserted into patient 105, an impedance measurement of a lead of the plurality of leads inserted into patient 105, or an evoked compound action potential (ECAP) signal sensing for the patient. Diagnostic unit 361 may output, with telemetry circuitry 358, an indication of diagnostic information associated with the diagnostic test to a remote device and/or a remote client.

Communication mode unit 363 may be configured to select a mode of communication (e.g., a text message, a phone call, a virtual appointment, an in-person appointment, etc.). For example, communication mode unit 363 may receive patient information from a medical device (e.g., IMD 110). Communication mode unit 363 may select a communication mode for the patient based on the patient information and/or medical device information for the first medical device. Communication mode unit 363 may exchange, with telemetry circuitry 358, data using the selected communication mode. In some examples, communication mode unit 363 may be omitted. For example, a remote device may select the mode of communication.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

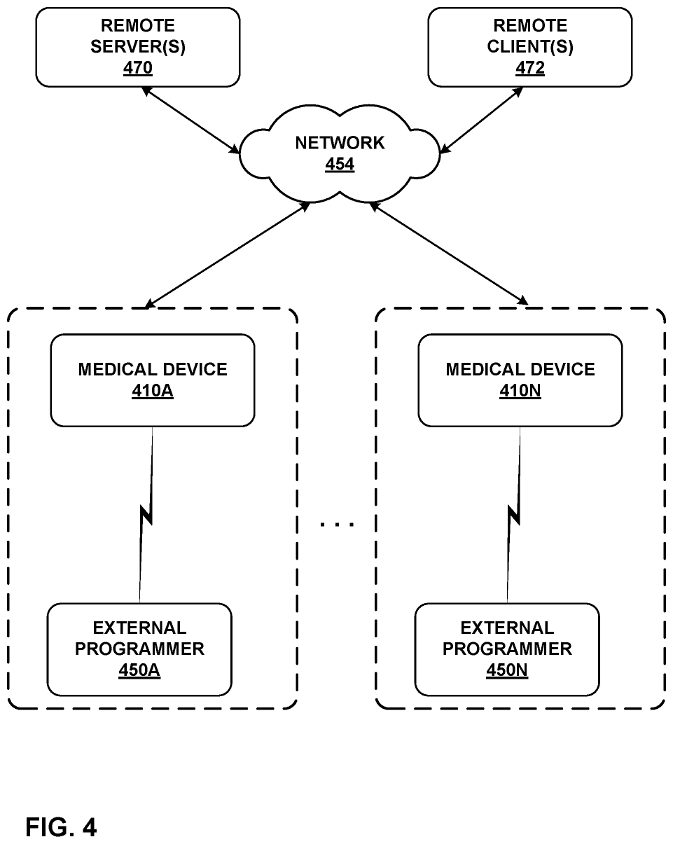

FIG. 4 is a block diagram illustrating an example of one or more remote servers 470 (referred to herein as “remote server 470”) and one or more remote clients 472 (referred to herein as “remote clients 472”) suitable for use with the IMD of FIG. 1, in accordance with one or more techniques of this disclosure. Remote server 470 may represent a cloud computing infrastructure, such as, for example a cloud or web interface. Remote client 472 may represent a clinician device geographically remote from external programmer 150 and/or IMD 110. In some examples, remote server 470 may work with remote client 472. For instance, remote server 470 may store data or at least partially process data for remote client 472. Remote client 472 may be used by a health professional at a doctor's office and the patient and IMD 110 may be at a home of the patient. Remote server 470 and/or remote client 472 may be referred to herein as a remote device. Network 454 may comprise one or more wired (e.g., Ethernet) and/or wireless networks (e.g., Wi-Fi™, Bluetooth™, Zigbee™, IEEE 802.11, etc.). In some examples, network 454 may comprise the Internet. While the previous examples refer to remote client 472 as performing various processes, any combination of medical devices, external programmers 450, remote server 470, or remote client 472 may perform such processes. Moreover, remote client 472 (or any combination of medical devices, external programmers 450, remote server 470, or remote client 472) may perform the processes described as being performed by external programmer 150 of FIG. 1 and/or external programmer 300 of FIG. 3.

A remote device (e.g., remote server 470 and/or remote client 472) may be configured to control IMD 110 with a program or a group of programs to provide stimulation. For example, the remote device may automatically or semi-automatically set or adjust programs at IMD 110. For instance, in response to a change in activity of a patient (e.g., standing, walking, voiding, etc.), the remote device may automatically or semi-automatically set or adjust programs at IMD 110. For instance, the remote device may receive sensor information or user input information from IMD 110 or external programmer 150 via the network 454 that indicates a change in activity of the patient. While the following examples refer to remote client 472 as performing processes directed to identifying problem patients, initiating a diagnostic test, prioritizing a delivery of data, scheduling a virtual appointments, any combination of medical devices 410A-410N (collectively, “medical devices 410”), external programmers 450A-450N (collectively, “external programmers 450”), remote server 470, or remote client 472 may perform processes described herein directed to identifying problem patients, initiating a diagnostic test, prioritizing a delivery of data, scheduling a virtual appointments.

Remote client 472 may provide a snapshot where a clinician can access centralized patient data. For example, the snapshot may allow the clinician to sort and/or filter a patient list on different parameters to see “interesting” patients. For instance, remote client 472 may provide the snapshot that, instead of flipping through views, allows a clinician to develop rules for highlighting interesting patients. In some examples, the snapshot may comprise one or more “cards”, where a card may be added to view with information on why a patient is interesting, a next action to take, and/or other information. In some examples, a card may include one or more of: when does patient need refill; whether or not the patient missed upload; when will a fill alarm expire; and/or information from an external patient programmer. Information from the external patient programmer patient may include, for example, a bolus use in a patient-controlled mode (PTM) device and/or whether or not the patient has used patient boluses (e.g., the patient may delay appointment).

Remote client 472 may be configured to compare the patient information from the external programmer to user-configurable thresholds (e.g., a date of when to notify clinician) such that user-configurable dates for notification. In this way, remote client 472 may help the snapshot be flexible for different patients and/or different clinicians and/or help to make some actions limited to a practice account manager (e.g., when refill moved out so, different people are not producing different results based on different skill levels). Remote client 472 may present the snapshot such that any individual patient page shows why a particular patient is interesting (e.g., in an upper left corner) and/or allows the patient to follow and unfollow the particular patient. Remote client 472 may present the snapshot to include a section on implants that shows an implant status (e.g., the patient may have two pumps where one pump is a replacement pump).

In some examples, remote client 472 may track changes via serial numbers such that remote client 472 may track information for filtering. For example, remote client 472 may map a serial number to a location (e.g., leads, catheters). In some examples, remote client 472 may track number of changes (e.g., parameters) via each serial number. In some examples, remote client 472 may, in identifying particular patients, add recommendation of other features that may be utilized or optimized (e.g., could be new, extra features or features for remediation). For example, remote client 472 may add to a patient review system (PRS), ECAPs, etc. as recommendations (e.g., if a patient is making many changes when moving to different postures). In this way, remote client 472 may identify new features to add and/or one or more refinement of features.

Remote client 472 may track a lead tip movement in relation to stimulation changes (e.g., a lead or an impedance issue) and/or run an impedance check or lead location check. remote client 472 may use one or more accelerometers to track changes relative to efficacy. For instance, remote client 472 may determine that changes relative to efficacy may correspond to too many changes and, in response to the determination, filter up the patient information. Remote client 472 may allow the clinician to configure a preset in preferences when the patient information is prioritized. Remote client 472 may use a rolling trend over time as threshold for a change (e.g., upgrade) in prioritization of the patient.

Remote client 472 may use one or more accelerometers in a pump, ambulatory or not ambulatory, as indication of activity of patient. For instance, laying down too much may indicate pain. Remote client 472 may use patient activity as an input to filter. For example, remote client 472 may determine that a relatively low patient activity (e.g., inactive) indicates a lack of efficacy of therapy. In contrast, remote client 472 may determine that a relatively high patient activity (e.g., very active) indicates opportunity to adjust to save power.

Remote client 472 may apply data-informed access to IMD data. For example, remote client 472 may reduce a frequency or adjust timing of data recovery based on patient status. Remote client 472 may step up frequency of monitoring if there is a problem or prioritization or just use normal monitoring when there is no problem or prioritization (e.g., but could prioritize or recommend for prioritization based on the information that is retrieved). Remote client 472 may refrain from triggering off of diary events, e.g., for pelvic health, based on the data-informed access to IMD data.

Remote client 472 may reduce cycling and/or amplitude. For example, remote client 472 may cause one of medical devices 410 to operate in a low energy mode if everything seems to be going well. Remote client 472 may, when using ECAPS, bin patients into over and under stimulation status. In this way, remote client 472 may determine how often a patient adjusts out of range.

Remote client 472 may automate a request to silence alarm. In some examples, remote client 472 may generate additional diagnostic tests on one or more of medical devices 410 as described further herein. Remote client 472 may present a snapshot that shows not just interesting patients, but interesting settings for patients (e.g., overuse or underuse of drug).

Remote client 472 may group multiple patients. For example, remote client 472 may present a snapshot that provides a population level analysis and/or visualization. For instance, remote client 472 may generate a patient population group with better or worse outcomes. Remote client 472 may group patients based on one or more of a physiologic response or patient attributes. Examples of patient attributes may include, for example, a program, a placement of a medical device, a disease state, a clinic, or other attributes. In some examples, remote client 472 may cause external programmers 450 to present one or more questions for a patient. For instance, remote client 472 may cause external programmer 450 to present “Are you on non-pump (systemic) meds in addition to pump meds?”.

In some examples, patient attributes may include multi-modal data inputs. Examples of multi-modal data inputs may include, for example, a scan bar code, a radio-frequency (RF) read, or camera identify process to identify a drug. In this example, remote client 472 may cause external programmers 450 to direct the patient to answer questions and/or collect other information, such as, for example, input from wearable devices.

Remote client 472 may be configured to apply a failover process where patient information is collected from medical devices 450 and in the case of one or more patient attributes being unavailable from medical devices 450, remote client 472 may attempt to collect the unavailable patient attributes from one or more wearable devices. In this example, in the case of one or more patient attributes being unavailable from both medical devices 450 and wearable device(s), remote client 472 may attempt to collect the unavailable patient attributes from patient-reported outcomes (PROs). In some examples, remote client 472 may combine data from this hierarchy (e.g., medical devices, then wearable devices, then PROs). In some examples, however, remote client 472 may use information from a single device (e.g., only a medical device, only a wearable device, only PROs).

In some examples, remote client 472 may perform a longitudinal data acquisition process. For example, remote client 472 may collect patient information (e.g., patient attributes) from different devices and across different device types (e.g., in hierarchy of devices). For instance, remote client 472 may collect a first set of attributes for a patient from medical device 450A, a second set of attributes for the patient from a wearable device associated with the patient, and a third set of attributes for the patient collected from PROs input into external programmer 450A.

Remote client 472 may change a modality of communication based on one or more of an event, one or more importance attributes (e.g., why an attribute is interesting), or a user configuration. For example, remote client 472 may change a modality of communication to one or more of an e-mail, a text, or communicate to different user like a healthcare professional (HCP) based on one or more importance attributes.

Remote client 472 may change an architecture to recommend or even automate changes to device settings of medical devices 410 and/or communication to the patient, such as, for example, silencing an alarm, request additional diagnostic (e.g., physiologic measures or impedance measures).

Remote client 472 may detect medical concerns. Examples of medical concerns may include, for example, one or more of a dose escalation, or a potential use of more stimulation than needed or the potential for a patient being drug naive after catheter issue.

In some examples, remote client 472 may track a number of times a patient changes parameters of the medical device associated with the patient. Examples of a patient change may include, for example, a stim-up operation, a stim-down operation, a change of a pulse-width (PW), or a rate. A change in pulse-width may be in microseconds (μs). Remote client 472 may recommend other features that are not being optimized or utilized. Remote client 472 may track what features are being used and how often the features are being used. Remote client 472 may track a lead tip movement. Remote client 472 may track a patient activity via a 3-axis accelerometer plot. The 3-axis accelerometer plot may be easily for a clinician to understand a patient activity and to apply changes to help yield efficacy. Remote client 472 may notify a physician if too many changes are occurring (e.g., a number of changes exceed a threshold value). In some instances, too many changes of a medical device may indicate a possible yield issue.

Remote client 472 may use accelerator information from an accelerometer arranged into a (e.g., a medical device configured for TDD) to track patient activity. Remote client 472 may track a body position via 3-axis accelerometer to understand more of the patient activity and/or patient sleep. In some examples, remote client 472 may track a body position via 3-axis accelerometer to determine whether the patient is bed ridden. Remote client 472 may use a weighting on patient attributes that is at least partly pre-configured with patient data, which may help a clinician decide which patient attributes to use for flagging.

Remote client 472 may comprise an on-board, local, or remote diagnostic system configured to run periodically, or in response to triggering, to identify problems with patients receiving therapy. Problems may be indicated by patient input, therapy usage patterns, and/or sensed signals or conditions (e.g., impedance, ECAPs, and/or pressure). Based on indicated problems, remote client 472 may run diagnostic tests (smart troubleshooting), e.g., on devices, leads, catheters, or other components, schedule appointments, and/or generate a series of questions to elicit patient input regarding the problem. As one example, lead integrity could be indicated by field spread measurements and/or presence or absence of ECAP signals.

Remote client 472 may perform one or more selection of diagnostic steps aided by machine learning of troubleshooting approaches taken for a large population of similarly situated patients. Remote client 472 may be configured based on clinician preference to identify to the clinician (e.g., by notification or presentation) particular patients having specified types or severity levels of problems, thereby prioritizing or filtering data. In this way, the clinician receives a “hot list” of patients experiencing particular problems or problems of a specified severity, while filtering out information the clinician does not need.

The clinician may specify a remediation plan (e.g., automated changes in therapy parameters, automated scheduling of appointments, additional monitoring) or escalation plan (e.g., browser presentation, email, text, call or personnel) that is automatically performed by the diagnostic system to eliminate the problem and/or escalate levels of attention to the problem by the clinician.

Remote client 472 may trigger one or more remediation plans and/or escalation plans by different problem types, severity levels, or other conditions tailored to the clinician or patient. In some examples, remote client 472 may only provide data to the clinician if a problem is identified, instead of always sending data.

By tailoring remediation, escalation, and prioritization of problems, a clinician has flexibility in managing the amount of information raised to their attention. This may be advantageous for limiting information consumed by high volume clinicians, with many patients, but also for highlighting significant problems presented to lower volume clinicians. In some examples, the clinician also may select the type and quantity of information available for viewing by patients.

Remote client 472 may use different risk control measures for different patients and different risk scenarios. Remote client 472 may assign different trust levels to different patients to permit them to make therapy adjustments or grant requests for remote therapy adjustments, e.g., based on different trust levels and/or different scenarios. Approval of therapy changes for some patients may be voluntary or based on website, email or text communication with a caregiver, whereas other patients may require live voice or video communication. Live communication may be more important for TDD therapies. In some examples, remote client 472 may mediate an audio or video communication (immediate or scheduled) between patient and caregiver to address an urgent scenario. Remote client 472 may use time-stamped audio or video snippets to observe patient condition and grant approval for patient adjustment or remote adjustment. Likewise, approval of therapy changes for different scenarios may be subject to different communication modes, such that one scenario may be handled with less urgent, less intrusive communication while another may be handled with different, more intrusive modalities.

In accordance with the techniques of the disclosure, one or more of external programmer 450A, remote server 470, or remote client 472 may determine a communication mode. While external programmer 450A is used as an example external programmer, any one of external programmers 150 may be used to determine a communication mode. Examples of communication modes may include, for example, one or more of a browser presentation, an e-mail, a text message, a call, a virtual visit, a video message, a voice message, or an in-person visit.

One or more of external programmer 450A, remote server 470, or remote client 472 may determine the communication mode based on patient information. For example, one or more of external programmer 450A, remote server 470, or remote client 472 may determine a severity level for patient information. For instance, each communication mode of a plurality of communication modes may assigned a respective range of severity levels. In this example, one or more of external programmer 450A, remote server 470, or remote client 472 may select the communication mode that comprise a range of severity level that includes the severity level for the patient information. For instance, patient information may be associated with a severity level of 3, which may be assigned to a text message. In this instance, one or more of external programmer 450A, remote server 470, or remote client 472 may select a text message as a communication mode for the patient information.

One or more of external programmer 450A, remote server 470, or remote client 472 may output patient information using a communication mode. In this way, a system (e.g., one or more of external programmer 450A, remote server 470, or remote client 472) may help to provide risk control measures that simplify or make changes to a therapy easier for low risk changes using simple and/or non-intrusive communication modes (e.g., an e-mail, a text message, an in-app notification, etc.) and that may alert or prioritize changes to a therapy that may be relatively risky and would most likely benefit from a clinician review using relatively complex communication modes (e.g., a telephone call, a virtual visit, an in-person visit, etc.), which may improve a therapy provided to the patient. Moreover, providing risk control measures using a communication mode may allow a clinician to more quickly review therapy changes, which may help to reduce an amount of time a clinician spends reviewing patient information.

Remote client 472 may instruct the patient to assume various postures and cause medical device 410A to sense ECAPs or other signals with each posture, e.g., to obtain a stimulation intensity vs. ECAPS growth curve for verification or adjustment of closed loop intensity control of stimulation (either automatically or by the clinician). Remote client 472 may cause medical device 410A to perform a lead integrity check (or catheter check), and/or may collect vital information such as an electrocardiogram (ECG), breathing rate, etc. Remote client 472 may generate clinician reminders (e.g., a drug refill) or follow-up tasks for patients. In this way, remote client 472 may provide a virtual assistant to schedule a virtual patient interaction with the patient to help to improve a therapy provided to the patient. Moreover, providing the virtual assistant may allow a clinician to more quickly review therapy changes, which may help to reduce an amount of time a clinician spends reviewing patient information.

In this way, the system may help to provide patient control measures that simplify or make changes to a therapy easier for a patient, which may improve a therapy provided to the patient. Moreover, patient control measures may reduce a number of times that a clinician sets an intensity level for therapy, which may help to reduce an amount of time a clinician spends configuring medical devices.

Although shown as separate entities, in some examples, functionality may be distributed differently than that shown in FIG. 4. For example, remote server 470 and remote client 472 may be the same system. While the previous examples refer to remote client 472 as performing various processes, any combination of medical devices, external programmers 450, remote server 470, or remote client 472 may perform such processes.

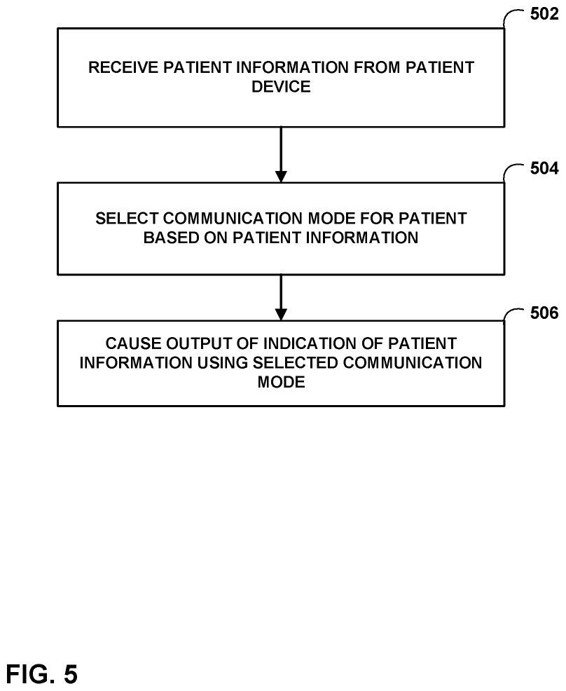

FIG. 5 is a flow diagram illustrating a process for selecting a communication mode to output patient information, in accordance with one or more techniques of this disclosure. FIG. 5 is discussed with reference to FIGS. 1-5 for example purposes only. In the following example, remote client 472 performs 502-506 of FIG. 5. However, in other examples, other devices may perform the process of FIG. 5 as explained in further detail below. In the following examples, IMD 110 is used as a medical device. However, in some examples, an external medical device may be used instead of IMD 110.

Remote client 472 may receive patient information from a medical device (502). The medical device may be configured to provide one or more of deep brain stimulation (DBS), spinal cord stimulation (SCS), sacral neuromodulation (SNS), or targeted drug delivery (TDD). Remote client 472 may select a communication mode for the first patient based on the patient information (504). For example, remote client 472 may select the communication mode to include one or more of a browser presentation, an e-mail, a text message, a call, a time-stamped audio snippet, a time-based video snippet, a virtual visit, or an in-person visit. Remote client 472 may cause an output of the indication of the patient information using the selected communication mode (506). For example, remote client 472 may display the patient information using one or more of a browser presentation, an e-mail, a text message, a call, a time-stamped audio snippet, a time-based video snippet. In some examples, remote client 472 may initiate (e.g., schedule or recommend) a virtual visit and/or an in-person visit.

For example, the medical device may comprise an implantable fluid delivery device. Remote client 472 may receive an indication of an amount of medication stored by a reservoir of a medical device (e.g., IMD 110). In this example, remote client 472 may select the communication mode for the patient based on the amount of medication stored by a reservoir of a medical device. For instance, remote client 472 may select a first communication mode when the amount of medication stored by the reservoir is less than a first threshold (e.g., an empty threshold), selecting a second communication mode when the amount of medication stored by the reservoir is greater than the first threshold and less than second threshold (e.g., a low threshold), and selecting a third communication mode when the amount of medication stored by the reservoir is greater than the first threshold.

The medical device comprise an implantable medical device configured to deliver electrical stimulation therapy. Remote client 472 may receive an indication of an energy level of an energy stored device for the medical device. In this example, remote client 472 may select the communication mode for the patient based on the energy level of the energy stored device for the medical device. For instance, remote client 472 may select a first communication mode when the energy level of the energy stored device is less than a first threshold (e.g., a discharged battery threshold), selecting a second communication mode when the energy level of the energy stored device is greater than the first threshold and less than second threshold (e.g., a low battery charge threshold), and selecting a third communication mode when the energy level of the energy stored device is greater than the first threshold.

Remote client 472 may determine a severity level based on the patient information. In some examples, the severity level may comprise one or more of an information level, a caution level, or a severe level. Each communication mode of a plurality of communication modes may comprise a respective range of severity levels. In this example, remote client 472 may determine the respective range of severity levels of the plurality of communication modes that includes the severity level for the patient information. For instance, patient information may be associated with a severity level of 3, which may be assigned to a text message. In this instance, remote client 472 may select a text message as a communication mode for the patient information.

Remote client 472 may determine the severity level using a tag. For example, remote client 472 may determine a tag associated with the patient information. In this example, remote client 472 may determine the severity level for the patient information as a severity level specified by the tag. The severity level specified by the tag may be preconfigured. In some examples, the severity level specified by the tag may be set by a clinician.

Remote client 472 may set the severity level specified by the tag based on feedback from one or more of the medical device or a user interaction. For example, remote client 472 may apply a machine learning algorithm trained using the feedback. In some examples, remote client 472 may train the machine learning algorithm using the feedback.

In some examples, remote client 472 may output an instruction to cause the medical device to perform a diagnostic test to generate diagnostic information. For example, remote client 472 may output the instruction to cause the medical device to perform one or more of a lead location diagnostic for a plurality of leads inserted into patient 105, an impedance measurement of a lead of the plurality of leads inserted into patient 105, or an ECAP signal sensing for the patient. Remote client 472 may receive an indication of the diagnostic information. In this example, remote client 472 may select the communication mode for the patient is based on the diagnostic information.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable gate arrays (FPGAs), or other equivalent integrated or discrete logic circuitry. Accordingly, the terms “processor” and “processing circuitry,” as used herein may refer to any of the foregoing structures or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

What is claimed is:

1. A method for managing communications with a patient, the method comprising:
receiving, by one or more processors, patient information for a medical device associated with the patient;
selecting, by the one or more processors, a communication mode for the patient based on the patient information; and
causing, by the one or more processors, an output of the indication of the patient information using the selected communication mode.

2. The method of claim 1, further comprising:
determining, by the one or more processors, a severity level based on the patient information;
wherein each communication mode of a plurality of communication modes comprises a respective range of severity levels; and
wherein selecting the communication mode comprises determining the respective range of severity levels of the plurality of communication modes that includes the severity level for the patient information.

3. The method of claim 2, wherein determining the severity level comprises:
determining a tag associated with the patient information; and
determining the severity level for the patient information as a severity level specified by the tag.

4. The method of claim 3, wherein the severity level specified by the tag is preconfigured.

5. The method of claim 3, wherein the severity level specified by the tag is set by a clinician.

6. The method of claim 3, further comprising setting the severity level specified by the tag based on feedback from one or more of the medical device or a user interaction.

7. The method of claim 6, wherein determining the severity level specified by the tag comprises applying a machine learning algorithm trained using the feedback.

8. The method of claim 7, further comprising training the machine learning algorithm using the feedback.

9. The method of claim 1,
wherein receiving the patient information comprises receiving an indication of an amount of medication stored by a reservoir of the medical device; and
wherein selecting the communication mode for the patient is based on the amount of medication stored by the reservoir of the medical device.

10. The method of claim 9, wherein selecting the communication mode comprises selecting a first communication mode when the amount of medication stored by the reservoir is less than a first threshold, selecting a second communication mode when the amount of medication stored by the reservoir is greater than the first threshold and less than second threshold, and selecting a third communication mode when the amount of medication stored by the reservoir is greater than the first threshold.

11. The method of claim 1, wherein the medical device includes an implantable fluid delivery device.

12. The method of claim 1,
wherein receiving the patient information comprises receiving an indication of an energy level of an energy stored device for the medical device; and
wherein selecting the communication mode for the patient comprises selecting the communication mode from a plurality of communications modes based on the energy level of the energy stored device for the medical device, wherein the plurality of communications modes comprises one or more of a browser presentation, an e-mail, a text message, a call, a virtual visit, a video message, a voice message, or an in-person visit.

13. The method of claim 12, wherein selecting the communication mode comprises selecting a first communication mode when the energy level of the energy stored device is less than a first threshold, selecting a second communication mode when the energy level of the energy stored device is greater than the first threshold and less than second threshold, and selecting a third communication mode when the energy level of the energy stored device is greater than the first threshold.

14. The method of claim 1, wherein the medical device includes an implantable medical device configured to deliver electrical stimulation therapy.

15. The method of claim 1, further comprising outputting, by the one or more processors, an instruction to cause the medical device to perform a diagnostic test to generate diagnostic information;
wherein receiving the patient information comprises receiving an indication of the diagnostic information; and
wherein selecting the communication mode for the patient is based on the diagnostic information.

16. The method of claim 15, wherein outputting the instruction comprises outputting the instruction to cause the medical device to perform one or more of a lead location diagnostic for a plurality of leads inserted into the patient, an impedance measurement of a lead of the plurality of leads inserted into the patient, or an evoked compound action potential (ECAP) signal sensing for the patient.

17. The method of claim 1, wherein selecting the communication mode comprise one or more of a browser presentation, an e-mail, a text message, a call, a time-stamped audio snippet, a time-based video snippet, a virtual visit, or an in-person visit.

18. The method of claim 1, wherein the medical device is configured to provide one or more of deep brain stimulation (DBS), spinal cord stimulation (SCS), sacral neuromodulation (SNS), or targeted drug delivery (TDD).

19. The method of claim 1, wherein the severity level comprises one or more of an information level, a caution level, or a severe level.

20. A device comprising:
memory; and
processing circuitry coupled to the memory, wherein the processing circuitry is configured to:
receive patient information for a medical device associated with the patient;
select a communication mode for the patient based on the patient information; and
cause an output of the indication of the patient information using the selected communication mode.
